(19) United States
(12) Patent Application Publication
(10) Pub. No.: US 2012/0214734 A1
(43) Pub. Date: Aug. 23, 2012

(54) TREATMENT OF METABOLIC DISORDERS

(75) Inventor: Paul Anthony TALBOT, Indianapolis, IN (US)

(73) Assignee: Paul A. Talbot, Indianapolis, IN (US)

(21) Appl. No.: 13/106,608

(22) Filed: May 12, 2011

Related U.S. Application Data
(63) Continuation of application No. 13/005,286, now abandoned.
(60) Provisional application No. 61/444,762, filed on Feb. 20, 2011.

(51) Int. Cl. A61K 38/05; A61P 3/00; A61P 3/06; A61P 3/04; A61P 9/12; A61K 31/401; A61P 3/10

(52) U.S. Cl. 514/4.8; 514/423; 514/6.8; 514/16.2; 514/16.3; 514/7.4

(57) ABSTRACT

The present invention provides methods for treating, preventing, and/or delaying the onset of metabolic disorders including multi-factorial syndromes such as insulin-insensitivity and metabolic syndrome by administering an effective amount of an ACE inhibitor.

This application claims priority from and the benefit of U.S. Non-Provisional Patent Application No. 13/005,286 filed 12 Jan. 2011 and U.S. Provisional Patent Application Ser. No. 61/444,762, filed 20 Feb. 2011.

### FIELD OF THE INVENTION

The invention relates to the fields of pharmacology and medicine, and provides methods for treating metabolic disorders including metabolic syndrome and insulin resistance.

### BACKGROUND

Disorders in lipid and carbohydrate metabolism are well-known abnormalities that increase the risk of diabetes and cardiac dysfunction, two of the most pressing medical concerns of modern society. A wide variety of metabolic diseases can arise from a multiplicity of underlying genetic and/or environmental causes.

A particularly insidious metabolic disorder, referred to as “insulin resistance,” is characterized by the body's impaired response to insulin. Insulin resistance has been correlated with the following observations: (1) elevated serum free fatty acids and cytokines, e.g., resistin and leptin (Mooradian A. D., Growth Horm. IGF Res. 11:Suppl A:S79-83, 2001; Ravussin, E. and Smith, S. R., Ann N.Y. Acad. Sci. 967:363-78, 2002)); (2) certain proteins such as calpains (Baier, L. J. et al., J. Clin. Invest. 106:819-21, 2000); (3) abnormal regulation of amylin and calcitonin gene-related peptide (CGRP) (Leighton, B. and Cooper, G. J., Nature 335:632-5, 1988; Haynes, J. M. et al. Diabetologia 40:256-61, 1997); (4) elevated levels of interleukin 6 (IL-6) and C-reactive protein (CRP) (Hak, A. E. et al., J. Clin. Endocrinol. Metab. 86:4398-405, 2001; Pickup, J. C. et al., Diabetologia 40:1286-92, 1997); and (5) elevated growth hormone during puberty (Dunger, D. B. and Cheetham, T. D., Horm. Res. 46:2-6, 1996; Halldin, M. U. et al., Clin. Endocrinol. (Oxf) 48:785-94, 1998). Other markers of insulin resistance include hypertension, hyperglycemia, hypertriglyceridemia, and low levels of high density lipoprotein cholesterol, HDL-C (Reaven, 1988).

A related metabolic disorder, referred to as Metabolic syndrome, or Syndrome X, is a complex multi-factorial condition accompanied by an assortment of abnormalities including hypertension, hypertriglyceridemia, hyperglycemia, low levels of HDL-C, and abdominal obesity. Grundy, S. M. Am J. Cardiol. 81: 18B-25B, 1998.

According to the World Health Organization (WHO) Guideline, metabolic syndrome is present if an individual manifests: a) hypertension (>140 mm Hg systolic or >90 mm Hg diastolic); (b) dyslipidemia, defined as elevated plasma triglycerides (150 mg/dL), and/or low high-density lipoprotein (HDL) cholesterol (<35 mg/dL in men, <39 mg/dL in women); 3) visceral obesity, defined as a high body mass index (BMI) (30 kg/m2) and/or a high waist-to-hip ratio (>0.90 in men, >0.85 in women); and 4) microalbuminuria (urinary albumin excretion rate of 20 g/min). See WHO-International Society of Hypertension Guidelines for the Management of Hypertension. Guidelines Subcommittee. J. Hypertens. 17:151-183, 1999.

According to the National Cholesterol Education Program (NCEP ATP III study) metabolic syndrome is diagnosed if three (3) or more of the following five (5) risk factors are present: (1) a waist circumference >102 cm (40 in) for men or >88 cm (37 in) for women; (2) a triglyceride level of 150 mg/dL; (3) an HDL cholesterol level <40 mg/dL for men or <50 mg/dL for women; (4) blood pressure >130/85 mm Hg; or (5) a fasting glucose >110 mg/dL. JAMA 285: 2486-2497, 2001.

Each of the metabolic disorders associated with metabolic syndrome are risk factors in their own right, and can promote atherosclerosis, cardiovascular disease, stroke, and other adverse health consequences. However, when present together, these factors are predictive of increased risk of cardiovascular disease and stroke.

Treating metabolic conditions of the type mentioned, whether alone or in combination, generally proceed at least initially with recommended lifestyle changes including weight loss, exercise, diet control, smoking cessation, etc. However, when life style changes are inadequate, medications are routinely incorporated into treatment strategies. Available medications for treating such metabolic disorders generally target a single condition and/or abnormality. For example, hypertension is generally treatable with lifestyle changes and/or with blood pressure reducing agents such as ACE inhibitors and beta-blockers.

Currently, however, there is no recognized single drug treatment that addresses the multiplicity of conditions associated with insulin resistance and/or metabolic syndrome.

There remains a need for improved therapies for the treatment of metabolic abnormalities and conditions in a patient whether in isolation or when associated with syndromes such as insulin insensitivity and/or metabolic syndrome. The present invention relates to the unexpected discovery that administration of an ACE-inhibitor provides an effective treatment to increase insulin sensitivity and rectify or normalize abnormal levels of metabolic markers including, but not limited to, serum triglyceride, serum glucose, and HDL-C, even in the absence of weight loss.

### SUMMARY OF THE DISCLOSURE

Certain variations of the present invention provide improved treatment, prevention, and/or delay in the onset of metabolic conditions such as hypertriglyceridemia, hyperglycemia, and low HDL-C by administering an effective amount of an ACE inhibitor.

The invention provides methods for treating and/or preventing, and/or delaying the onset or progression in a patient of one or more metabolic conditions including hypertriglyceridemia, hyperglycemia, and/or low serum HDL-C by administering an ACE inhibitor.

In one embodiment, the invention provides methods for treating and/or preventing, and/or delaying the onset or progression of metabolic conditions in isolation or in plurality including as part of the metabolic syndrome and/or insulin resistance, comprising administering to a patient in need thereof of an effective amount of an ACE inhibitor.

This Summary is provided merely to introduce certain concepts, and is not intended to identify any key or essential features of the claimed subject matter.

### BRIEF DESCRIPTION OF THE DRAWINGS

FIG. 1 shows a correlation between waist-to-height and BMI in obese individuals.

FIG. 2A shows an age-related increase in HDL-C in obese individuals.

FIG. 2B shows and age-related increase in serum glucose in obese individuals.

FIG. 2C shows a direct correlation between random serum glucose and hypertriglyceridemia in obese individuals.

FIG. 3 shows the effect of administering Captopril on glucose, triglyceride, HDL-C, bodyweight, and blood pressure, with administration beginning at 24 months.

FIG. 4A shows the beneficial effects of ACE inhibitor over time on serum HDL-C levels.

FIG. 4B shows the age-related effect of ACE inhibitor on random serum glucose in obese individuals.

FIG. 4C shows the age-related effect of ACE inhibitor on triglyceride levels in obese individuals.

FIG. 5A shows the effect of ACE inhibitor administered alone or in combination with Hctz on glucose and triglyceride levels.

FIG. 5B shows the effect of Hctz on the age-related increase of HDL-C by ACE inhibitor.

FIG. 5C shows the effect of Hctz on age-related glucose level increase.

FIG. 6A shows the dose-effect of Lisinopril on HDL-C.

FIG. 6B shows the dose-effect of Captopril on triglyceride.

FIG. 7 shows the effects of Lisinopril, Captopril, and Enalapril on glucose, triglyceride, and HDL-C in obese patients.

FIG. 8A shows the effect of Simvastatin in combination with Captopril on glucose and HDL-C.

FIG. 8B shows the effect of Simvastatin in combination with ACE inhibitor on random serum HDL-C.

### DETAILED DESCRIPTION

As used herein the term “metabolic syndrome” or “syndrome X” refers to a clinically-recognized multi-factorial metabolic disorder/condition characterized by a plurality of metabolic conditions including a subset or all of hypertension, dyslipidemia, hyperglycemia, and visceral obesity.

As used herein “BMI” or “body-mass index” refers to an individual's body weight divided by the square of his or her height.

As used herein the terms “metabolic condition(s)” or “metabolic disorder(s)” refer generally to one or more abnormal or otherwise deficient metabolic function(s) and/or physiological state(s) that may be present in a patient individually or as a collection of more than one abnormality. In some cases a plurality of metabolic conditions occur as part of a recognized disease, condition, or syndrome. Exemplary, but not exclusive, metabolic conditions include abnormal lipid metabolic conditions including, for example, one or more of the following: hypertriglyceridemia, high total cholesterol, high LDL, and low HDL, for example low HDL-C. Other exemplary metabolic conditions include abnormal glucose metabolic function, and/or status including, for example, type-2 diabetes, glucose intolerance, elevated fasting glucose, elevated 2-hour glucose levels, and insulin-insensitivity.

As used herein, the terms “above-normal” or “below-normal” are applied in reference to some parameter or measure for which normal and/or desirable values and/or ranges are known and generally accepted by the relevant scientific and/or medical community as defining a normal or desirable value, or range of values to reduce the risk of disease. In the present context, these and similar terms may be applied to values and/or a range of values for metabolic substrates in the body, including, for example, lipids, lipoproteins, proteins, carbohydrates, and/or sugars that may be diagnostic and/or indicative of the state of health and/or certain underlying metabolic conditions or abnormalities.

As used herein, the term “insulin resistance” refers to a metabolic condition that may be associated with hypertension, hyperglycemia, hypertriglyceridemia, and low HDL-C. Insulin resistance impairs the body's ability to respond properly to insulin.

As used herein the term “ACE inhibitor,” or “angiotensin converting enzyme inhibitor,” refers to a class of antihypertensive compounds that act on the renin system to control blood pressure. Exemplary but non-exclusive ACE inhibitors include those disclosed herein. Many but not necessarily all ACE inhibitors have been approved for commercialization and are available as marketed products.

As used herein, the term “hyperglycemia” means high blood sugar, a condition in which an excessive amount of glucose circulates in the blood plasma. Generally, a glucose level higher than 10 mmol/l (180 mg/dl) is considered excessive. Chronically high levels of glucose exceeding 7 mmol/l (125 mg/dl) can produce organ damage. Glucose levels generally fluctuate before and after meals, and at various times of day. While the definition of “normal” varies among medical professionals, in general, the normal range of glucose for most people is about 80 to 110 mg/dl, or 4 to 6 mmol/1 (fasting adult). An individual with a consistent level above 126 mg/dl is generally held to have hyperglycemia.

As used herein, the term “hyperlipidemia” means the condition of abnormally elevated levels of any or all lipids and/or lipoproteins in the blood. It is the most common form of “dyslipidemia”, which also includes any decreased lipid levels. Hyperlipidemias are divided into primary and secondary subtypes. Primary hyperlipidemia is generally genetically-based, while secondary hyperlipidemia frequently arises in consequence of other causes, such as diabetes.

As used herein the term “obese” or “obesity” refers to the commonly understood medical condition in which excess body fat has accumulated, as defined or recognized by any suitable standard of measure known to the skilled artisan including, for example, body mass index (BMI), waist-hip ratio, and waist-height ratio. For example, according to the World Health Organization (WHO) a BMI between 25-29.9 is defined as overweight; 30-34.9 as class I obesity; 35-39.9 as class II obesity; and greater than 40 as class III obesity. Other classifications define a BMI of 35-40 as severe obesity; up to 49.9 as morbid obesity; and greater than 50 as super obesity. Another type of obesity, referred to as “abdominal obesity” or “visceral obesity”, or similar terms apply to the abdominal region and may be defined by the waist-to-hip ratio (>0.9 in men; >0.85 in women); alternatively as a BMI in excess of about 30 kg/m2. Abdominal obesity is one of the characteristics of the metabolic syndrome.

As used herein, “administer” or “administering” means to introduce, such as to introduce to a subject a compound(s) or composition. The term is not limited to any specific mode of delivery, and can include, but is not limited to subcutaneous, intravenous, intramuscular, intracisternal, delivery by infusion techniques, transdermal, oral, nasal, pulmonary, and rectal delivery.

As used herein, “treat” or “treating” or “treatment” refer to ameliorating and/or curing one or more symptoms characteristic of a disease, disorder, or condition.

As used herein, the term “prevent” refers generally to preventing a disease or condition from manifesting, or preventing a disease or condition from progressing and/or worsening in some clinically recognizable fashion.

As used herein, the phrase “delay the onset” or “delay the onset of” refer to delaying or extending the time at which a disease or condition, including symptom(s) thereof, manifest in a clinically recognizable fashion.

As used herein, “patient” refers to a human of either sex, but also may include other mammals such as horses, cows, sheep, pigs, mice, rats, dogs, cats, and primates other than humans. Patients may be obese or non-obese. In some embodiments, a patient may manifest one or more metabolic conditions and/or visceral obesity without being obese.

As used herein, the term “in combination with” refers to a mode of administration that places no limit on the method, mode, form, etc. of the administration. For example, “in combination with” would include, but is not limited to, simultaneous administration of two or more compounds, alone as single agents, or in a single composition; it would also include sequential administration of two or more compounds.

As used herein, the term “effective amount” or “therapeutically effective amount” refers to an amount of an active agent, e.g. ACE inhibitor that elicits a desired clinical response, for example, reduction or alleviation of the symptoms and/or conditions of a disease being treated. In some embodiments of the invention, the amount of active agent administered may vary depending on various factors, including but not limited to, the weight of a patient, the nature and/or extent of the patient's disease, etc.

### ACE Inhibitor Compounds

The invention relates to the administration of ACE inhibitor to treat, prevent, and/or delay the onset or progression of a metabolic condition or syndrome, including but not limited to hypertriglyceridemia, hyperglycemia, low HDL-C, insulin insensitivity, and metabolic syndrome. ACE inhibitors are synthetic compounds classified into 3 broad groups: (1) sulfhydryl-containing ACE inhibitors related to captopril, (2) dicarboxyl-containing ACE inhibitors related to enalapril (e.g. lisinopril), (3) phosphorus-containing ACE inhibitors related to fosinopril. All ACE inhibitors effectively block conversion of angiotensin I to angiotensin II through mimicry of the peptide angiotensin I. However, they differ in potency, pharmacokinetics, and whether or not they are pro-drugs requiring in vivo activation (Jackson, 2001).

Methods of the present invention comprehend use of any compound having ACE inhibitor activity, or any solvate, hydrate, pro-drug, polymorph, composition, or pharmaceutically acceptable salt thereof. Examples of pharmaceutically acceptable salts may include the sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, beta-hydroxybutyrate, glycollate, tartrate, methanesulfonate, propanesulfonate, naphthalene-1-sulfonate, naphthalene-2-sulfonate, mandelate, and the like.

The suitability of any particular compound as an ACE inhibitor can be readily determined by evaluation of potency and/or selectivity using methods known to the skilled artisan in accordance with standard pharmaceutical practice.

Preferred ACE inhibitors for use according to the present invention include the following commercially available products: Benazepril (Lotensin®), Captopril (Capoten®), Enalapril (Vasotec®), Fosinopril (Monopril®), Lisinopril (Prinivil®, Zestril®), Moexipril (Univasc®), Perindopril (Aceon®), Quinapril (Accupril®), Ramipril (Altace®), and Trandolapril (Mavik®)

It should be understood that use of a single ACE inhibitor is preferred by the present invention, but combinations of two or more ACE inhibitors may be desirable in certain cases as determined by the treating physician, and combinations of ACE inhibitors is within the scope of the invention.

As described in further detail below, it has now been unexpectedly discovered that administering an effective amount of an ACE inhibitor to a patient with or without hypertension provides therapeutic benefit for treating, preventing, and/or delaying the onset or progression of one or more metabolic conditions including hypertriglyceridemia, hyperglycemia, and low HDL-C. In particular, the invention relates to administering an ACE inhibitor to reduce elevated levels of triglyceride and/or glucose, and/or to increase levels of HDL-C in a patient in need thereof.

Administering ACE inhibitor to Treat, Prevent, and/or Delay Onset of a Metabolic Condition

In one embodiment, the present invention relates to administering an effective amount of an ACE inhibitor to a patient with or without hypertension to treat, prevent, or delay the onset or progression of one or more metabolic conditions, disorders, or syndromes.

A patient may be non-obese or obese, as defined herein.

In one embodiment, a patient has normal blood pressure but exhibits one or more risk factors and/or metabolic abnormalities including hypertriglyceridemia, hyperglycemia, low HDL-C and/or visceral obesity.

In another embodiment, a patient has elevated blood pressure and one or more other risk factors and/or metabolic abnormalities including hypertriglyceridemia, hyperglycemia, low HDL-C, and/or visceral obesity.

In another embodiment, a patient has one or more risk factors or metabolic markers for having, or for developing a metabolic condition and/or syndrome including insulin insensitivity and metabolic syndrome. For example, a patient may have a family history of one or more metabolic conditions or diseases, including, but not limited to, abnormal metabolic markers of carbohydrate and/or lipid metabolism, diabetes, cardiovascular disease, and/or stroke.

In another embodiment of the invention, a patient has less than the number of risk factors that support a diagnosis of insulin insensitivity and/or metabolic syndrome by an accepted medical standard including, for example, the standards established by WHO, or the NCEP ATP III Study.

In another embodiment of the invention, an ACE inhibitor is administered to treat a patient who has any one of the metabolic abnormalities and/or diagnostic features of a metabolic disease or syndrome selected from hypertension, hypertriglyceridemia, hyperglycemia, low HDL-C, and abdominal obesity.

In another embodiment, a patient has any two of the metabolic abnormalities and/or diagnostic features selected from hypertension, hypertriglyceridemia, hyperglycemia, low HDL-C, and abdominal obesity. In one aspect of this embodiment, at least one of the two abnormalities is hypertension. In another aspect, neither of the two abnormalities is hypertension.

In another embodiment, a patient has any three of the metabolic abnormalities and/or diagnostic features selected from hypertension, hypertriglyceridemia, hyperglycemia, low HDL-C, and abdominal obesity. In one aspect of this embodiment, the patient also has insulin insensitivity. In another aspect of this embodiment, the patient also has metabolic syndrome. In another aspect of this embodiment, at least one of the three abnormalities is hypertension. In another aspect, none of the three abnormalities is hypertension.

In another embodiment, a patient has any four of the metabolic abnormalities and/or diagnostic features selected from hypertension, hypertriglyceridemia, hyperglycemia, low HDL-C, and abdominal obesity. In one aspect of this embodiment, the patient also has insulin insensitivity. In another aspect of this embodiment, the patient also has metabolic syndrome. In a further aspect of this embodiment, at least one of the four abnormalities is hypertension. In another aspect, none of the four abnormalities is hypertension.

In another embodiment, a patient manifests multiple metabolic abnormalities and/or diagnostic features including hypertension, hypertriglyceridemia, hyperglycemia, low HDL-C, and abdominal obesity. In one aspect of this embodiment, the patient also manifests insulin insensitivity. In another aspect of this embodiment, the patient also has metabolic syndrome.

In another embodiment, a patient presents with normal levels of glucose and/or lipids such as triglyceride, and/or total cholesterol, and/or LDL, but has low HDL-C, with or without concomitant hypertension.

According to another embodiment of a method of the present invention, lifestyle changes may optionally be included as part of a treatment regimen with ACE inhibitor. Lifestyle changes may include but are not limited to weight reduction, increased exercise, diet control and smoking cessation.

In another aspect, a method of the present invention does not include requisite lifestyle changes. For example, an obese patient may be successfully treated with an ACE inhibitor without manifesting or requiring weight loss.

In another embodiment, the present invention relates to administering ACE inhibitor to a patient suffering from or at risk of developing one or more metabolic conditions selected from hypertension, hyperglycemia, hyperlipidemia, dyslipidemia, hypertriglyceridemia, atherosclerosis, insulin insensitivity, type 2 diabetes, impaired glucose tolerance, and metabolic syndrome.

In another embodiment, the method of the present invention relates to administering an effective amount of an ACE inhibitor to reduce serum triglyceride in a patient in need thereof.

In another embodiment, the method relates to administering an effective amount of an ACE inhibitor to reduce serum glucose in a patient in need thereof.

In another embodiment, the method relates to administering an effective amount of an ACE inhibitor to elevate serum HDL-C levels in a patient in need thereof.

In another embodiment, the method of the present invention relates to administering an effective amount of an ACE inhibitor to treat one or more metabolically abnormal conditions in a patient including hyperglycemia, hyperlipidemia, dyslipidemia, hypertriglyceridemia, atherosclerosis, insulin insensitivity, type 2 diabetes, and impaired glucose tolerance where the patient does not qualify for a diagnosis of metabolic syndrome.

Most, if not all, ACE inhibitor compounds within the scope of the present invention are orally available, and oral administration is preferred. However, other routes of administration are also within the scope of the invention including, for example, transdermal, buccal, percutaneous, intravenous, intramuscular, intranasal, or intrarectal route. The route of administration may be varied in any way, limited only by the physical properties of the drugs and the convenience to the patient and/or caregiver.

In another embodiment of the present invention, an ACE inhibitor is co-administered with one or more other compounds including other suitable anti-hypertensive agents. Other anti-hypertensive agents include hydrochlorothiazide, metoprolol, atenolol, amlodipine, furosemide, hydralazine, propranolol, nifedipine, and verapamil.

In another embodiment, an ACE inhibitor is co-administered with an anti-lipemic agent. Anti-lipemic agents that may be co-administered with an ACE inhibitor include, but are not limited to, statins (e.g. Lovastatin®; Pravastatin®), bile acid sequestrants (Cholestyramine®; Colestipol®; Colesevelam®), nicotinic acid (Niaspan®), and fibric acids (Gemfibrozil®; Clofibrate®).

When co-administering an ACE inhibitor and other agent, administration may be concomitant or sequential, that is separately in time in a sequential manner, wherein one compound is administered first and the other second, or vice versa. Sequential administration may be close in time or remote in time. For example, ACE inhibitor and another agent can be administered about 30 minutes apart, or about 1 hour apart, or about 2 hours apart, or about 4 hours apart, or about 8 hours apart, or about 12 hours apart, where ACE inhibitor is administered earlier than the other agent, or vice versa.

Dosages of ACE inhibitor according to the present invention must, in the final analysis, be set by the attending physician. General outlines of suitable dosages are provided below. Generally, a suitable dose of an ACE inhibitor, or a pharmaceutically acceptable salt thereof, or composition thereof, for administration to a human will be in the range of about 0.5 mg/day to about 1000 mg/day; preferably, from about 1 mg/day to about 500 mg/day. Dosage guidelines for specific ACE inhibitors are as follows:
Benazepril: from about 10 mg/day to about 80 mg/day; preferably, from about 10 mg/day to about 40 mg/day.
Captopril: from about 50 mg/day to about 450 mg/day; preferably, from about 12.5 mg/day to about 25 mg 2-3 times/day.
Enalapril: from about 2.5 mg/day to about 40 mg/day.
Fosinopril: from about 10 mg/day to about 80 mg/day; preferably, from about 20 mg/day to about 40 mg/day.
Lisinopril: from about 10 mg/day to about 80 mg/day.
Moexipril: from about 5 mg/day to about 30 mg/day.
Perindopril: from about 4 mg/day to about 16 mg/day.
Quinapril: from about 10 mg/day to about 80 mg/day.
Ramipril: from about 2.5 mg/day to about 20 mg/day.
Trandolapril: from about 1 mg/day to about 8 mg/day.
Cilazapril: from 2.5 mg/day to about 10 mg/day.

In certain embodiments, a therapeutic method according to the present invention may include one or more additional steps. In one embodiment of this aspect, the metabolic status of a patient and/or the effectiveness of treatment with ACE inhibitor is evaluated on one or more relevant markers. For example, a patient may be evaluated for one or more metabolic markers before, during and/or after initiation of treatment with ACE inhibitor.

In one embodiment, particular markers for a metabolic condition(s), for example, compound(s) and/or metabolite(s) are determined prior to initiating treatment with an ACE inhibitor. For example, a sample drawn from a patient, e.g. serum, is analyzed to determine levels of one or more compounds including, but not limited to, triglyceride, cholesterol, HDL, LDL, and glucose. If an abnormal/undesirable level of any one or more such metabolic compounds is detected the patient is a candidate for ACE inhibitor therapy, whether or not hypertension is also present.

In one embodiment of this aspect, particular compound(s) and/or metabolite(s) are measured or otherwise evaluated in a patient during, and/or after treatment with ACE inhibitor to evaluate the effectiveness of treatment. For example, changes in the level of one or more serum markers may be determined during the course of treatment at regular or irregular intervals, for example, daily, weekly, monthly, semi-annually, annually, or any combination thereof as deemed appropriate by the physician.

In one embodiment, the level of one or more relevant lipid(s), sugar(s), protein(s), or lipoprotein(s) is determined by measuring or otherwise evaluating the levels thereof in patient serum before and/or during treatment.

In certain embodiments, serum and/or other body samples from a patient are withdrawn or collected for evaluation of lipid panels and/or one or more other metabolic markers including, but not limited to, glucose, total cholesterol, LDL, LDL-C, triglyceride, and HDL-C, wherein a lowering of serum glucose and/or triglyceride, and/or an elevation of HDL-C is observed by treatment with an ACE inhibitor. Serum glucose may be evaluated, for example, as a random measurement, isolated fasting glucose, or by a glucose tolerance test.

Administration of an ACE inhibitor according to the present invention results in prevention, treatment, and/or delay in the onset of one or more metabolic conditions selected from hyperglycemia, hyperlipidemia, dyslipidemia, hypertriglyceridemia, low HDL-C, atherosclerosis, insulin insensitivity, type-2 diabetes, impaired glucose tolerance, insulin insensitivity, and metabolic syndrome. Treatment according to the present invention results in clinical benefits to the patient including, but not limited to, one or more of lowering triglyceride, lowering glucose, and elevating HDL-C levels.

Significantly, clinical benefits of the present methods are realized by the present invention even in the absence of weight loss. This can be a significant advantage to obese patients who cannot, or will not, lose weight, which is widely regarded as an effective lifestyle change that mitigates risk of metabolic abnormalities and cardiovascular disease.

Without intending to be bound by any particular theory, it is believed that low HDL-C is an early, if not the earliest, and most sensitive indicator or prognosticator of the insulin-resistant state. In general, HDL-C levels drop with age. If a patient is tested and found to have low HDL-C, regardless of the presence or absence of other metabolic risk markers including, but not limited to, hypertension, another aspect of the invention relates to the initiation of treatment with an ACE inhibitor to treat, prevent, and/or delay the onset of one or more further metabolic conditions and/or complications including insulin-resistance and/or metabolic syndrome.

Treatment according to the methods of the present invention raises HDL-C levels and thereby reduces the risk and/or delays the onset of later complications and/or diseases including dyslipidemia, hyperglycemia, insulin resistance, metabolic syndrome, cardiovascular disease, and stroke.

Therefore, in another aspect, a method according to the present invention relates to administering an effective amount of an ACE inhibitor to a patient having or believed to have low HDL-C, and measuring an elevation of HDL-C after initiation of treatment. For example, an increase in HDL-C may be observed within a period of weeks, months, or years after the initiation of treatment.

### Pharmaceutical Compositions

In preferred aspects of the invention, an ACE inhibitor is formulated alone or in combination with one or more other active ingredients in a pharmaceutical composition. As used herein, the term “pharmaceutical composition” refers to a liquid or solid composition, preferably solid (e.g., a granulated powder) that contains a pharmaceutically active ingredient (e.g. ACE inhibitor) and at least a carrier, diluent, or excipient, according to known methods of formulation.

In general, compositions contain from about 0.5% to about 50% of the active compounds in total, depending on the desired doses and the type of composition to be used. The amount of the compounds, however, is best defined as the effective amount, that is, the amount of each compound which provides the desired dose to the patient in need of such treatment.

In any embodiment where an ACE inhibitor and another active agent e.g. hydrochlorthiazide, are included in a pharmaceutical composition, such pharmaceutical compositions are preferably in a form suitable for oral use, for example, as tablets, troches, lozenges, aqueous, or oily suspensions, dispersible powders or granules, emulsions, hard or soft capsules, or syrups or elixirs.

Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example corn starch or alginic acid; binding agents, for example, starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, to form osmotic therapeutic tablets for controlled release.

Pharmaceutical compositions suitable for use in the present invention can be packaged and/or administered in various dosage forms for oral, rectal, nasal, pulmonary, topical (including transdermal, buccal, and sublingual), vaginal, or parenteral (including subcutaneous, intramuscular, intravenous, and intradermal) administration. Preferably, compositions are in a form for oral administration as discrete units (i.e., dosage forms), such as capsules, tablets, sachets, and the like. Preparation of compositions in forms intended for oral administration, preferably solid forms, is within the ability of one skilled in the art, including the selection of pharmaceutically acceptable additional ingredients from the groups listed above in order to provide pharmaceutically acceptable preparations. Such pharmaceutical compositions may be prepared by methods known in the pharmaceutical formulation art, for example, see Remington's Pharmaceutical Sciences, 18th Ed., (Mack Publishing Company, Easton, Pa., 1990).

The following examples are provided as illustrations of some embodiments of the invention and are not intended to limit the scope of the claimed subject matter in any way.

### Example 1

### Treatment of Metabolic Conditions with ACE Inhibitors

One-hundred ten (110) patient charts in chronic care for hypertension at an 1800-capacity prison were initially screened. The data included age, weight, height, ethnicity, medications, lipid panel results for glucose, triglycerides and HDL-C, and blood pressure. All known or previously diagnosed diabetics were excluded from the study. At least three chemical classes of ACE inhibitors were prescribed for the management of hypertension in the screened patients: enalapril, lisinopril, and captopril. In two charts, the patients were not hypertensive, and were not on a blood pressure control regimen. The frequency of use of various antihypertensive medications used in 70 of the screened charts were as follows: Hydrochlorothiazide (hereinafter “Hctz”) (75% with 11% as hctz:triamterene combination medication), ACE inhibitors (53%), metoprolol (17%), atenolol (16%), amlodipine (14%), furosemide (9%), hydralazine (7%), propranolol (3%), nifedipine (3%), verapamil (1%), diltiazem (1%), minoxidil (1%), a-methyldopa (1%), terazosin (1%), spironolactone (1%), clonidine (1%). The most frequent antihypertensive agents in this study were Hctz and ACE inhibitors. Anti-lipemic agents (simvastatin, gemfibrozil, and niacin) comprised 21% of the 110 screened charts. The average dose of simvastatin was 20 mg/day with a range of 10-40 mg/day.

The relationship between body mass index (BMI) and dyslipidemia is well known and is most evident at a BMI >25 (Denke et al, 1993). Consequently, only males with BMIs >26.29 kg/m2 were followed in this study, which reduced the number of evaluated charts to 105. Waist circumference measurements were obtained on 32 individuals who exhibited truncal obesity. A waist-to-height ratio (WHtR) was determined as a percentage, with a range of waist circumferences from 36.5-80 inches, and a mean of 47.1 inches. The WHtR range was 52.8-115.9%, with a mean of 67.0%. According to WHO, males having WHtR >53.6% are at increased risk for cardiovascular disease. Mean BMI+/−standard error of the mean (SEM) of screened charts was 33.1 kg/m2+/−0.5 (number of charts, n=105).

Several months after the start of the initial screen, 17 additional charts were added, 3 of which were not hypertensive. With respect to data analysis of Hctz, 9 additional charts were screened of which 2 had no Hctz, ACE inhibitor, or anti-lipemic agent (increasing n to 6), and 7 had Hctz but no ACE-I or anti-lipemic (increasing n to 15). For cases involving ACE inhibitor plus simvastatin, further analysis included evaluation of total cholesterol and calculated low density lipoprotein (LDL-C).

In order to evaluate the effects of simvastatin without ACE inhibitor, 5 additional charts were screened (increasing n to 13). The average daily dose of simvastatin was 40 mg/day with a range of 20-80 mg/day. BMI range and mean+/−SEM for the total additional charts (n=17, no diabetics) were 27.1-41.9 kg/m2 and 32.4+/−1.1 kg/m2, respectively.

The data were analyzed with respect to random serum lab values including random serum glucose and fasting lipid panel (which included total cholesterol, HDL-C, TG, and the calculated LDL-C). These data were plotted against age and BMI and analyzed for effects of medications mainly Hctz, the anti-lipemic agents, and the ACE inhibitor. In addition, a few charts were evaluated for temporal changes spanning chronic care visits including weight, mean arterial blood pressure, medication adjustments, and random serum values of HDL-C, triglycerides, and glucose. A two-tailed student t test was used to test for significance between 2 sample means. Linear regression analysis with F statistic was used to obtain goodness of fit. Only coefficients of determination (r2) of 0.1 or greater were reported. Comparison of regression slopes was computed, and at statistic was applied to detect significant slope differences (Dixon, 1969). The level of significance used was a probability (P)<0.05.

### Results

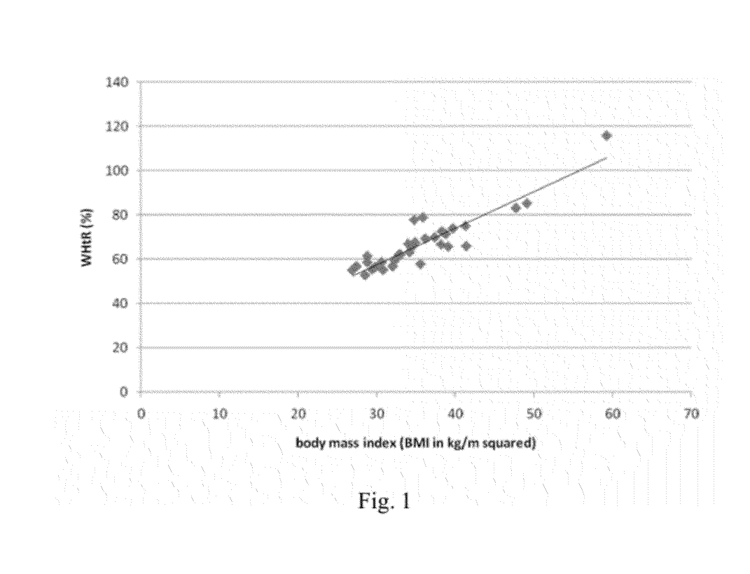

A significant (P<0.0001) direct correlation was observed between WHtR versus BMI (FIG. 1). Mean BMI and WHtR+/−SEM (n=32) were 35.7 kg/m2+/−1.2, and 67.0%+/−2.2, respectively. There was no correlation between random serum values (glucose, triglyceride, HDL-C) and the degree of obesity in this study. Four of 105 charts with largest BMI were in relatively young patients. The data collected for these 4 individuals, including age (years)/BMI (kg/m2)/glucose (mg/dl)/triglyceride (mg/dl)/HDL-C (mg/dl), was as follows: 30/59.2/98/102/33; 36/49.1/95/120/32; 35/47.7/88/86/44; 27/46.3/86/173/31. None of these 4 patients was on a anti-lipemic agent. Two of the 4 were on an ACE inhibitor (enalapril 10 mg/day in the 36 y/o, and enalapril 20 mg/day in the 27 y/o). These patients, despite their extreme obesity were not hyperglycemic, i.e. none had a serum glucose >100 mg/dl. However, 3 out of 4 had an HDL-C<40 mg/dl. For example, the largest WHtR was 115.9% in one of the 4, a 30-year old man, morbidly obese, not hypertensive, and on no chronic care medications or psychiatric medications. This individual's random serum glucose was 98 mg/dl and his 1 hr glucose tolerance test was normal at 106 mg/dl (normal range 65-159 mg/dl). Moreover, despite morbid obesity and a positive family history of diabetes (mother) the patient was not glucose intolerant. The only positive chemical marker detected in this patient for the insulin resistant state or metabolic syndrome was a low HDL-C (33 mg/dl). Consequently, it is hypothesized that low HDL-C, not hyperglycemia, is the earliest marker for the progression of insulin insensitivity.

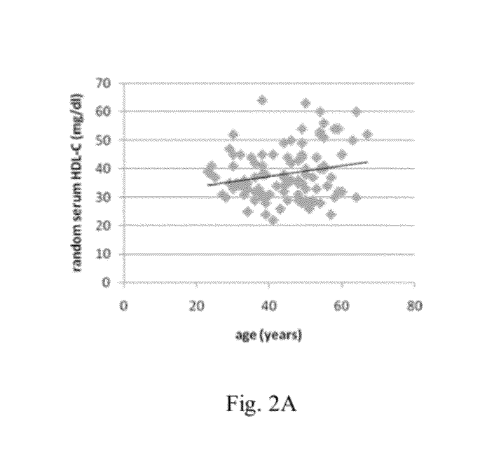

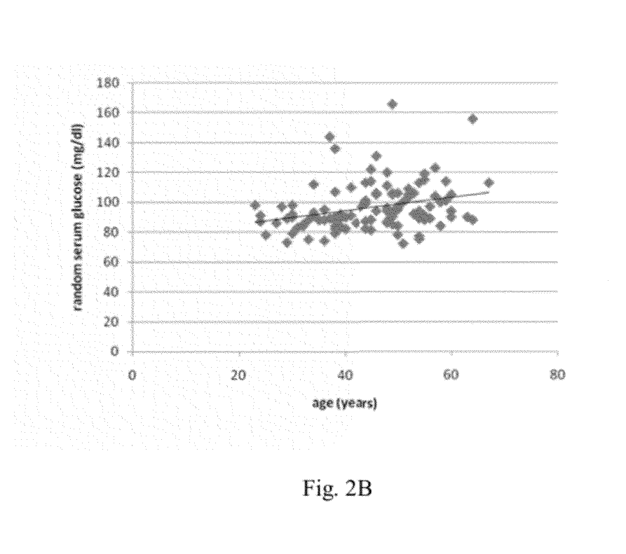

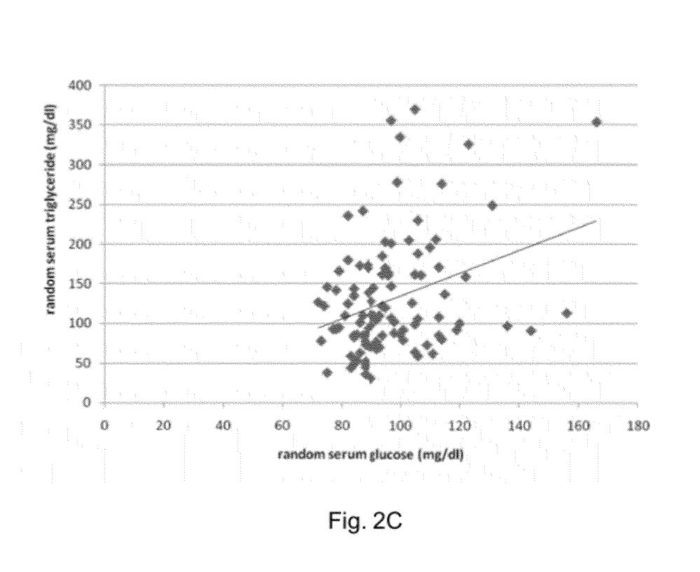

By evaluating scatter grams of the data, significant increases in random serum HDL-C, and glucose occurred with age (FIG. 2A, 2B, respectively). There was a trend towards an increase in random serum triglyceride with age. However, a significant (P<0.001) and direct correlation existed between age-related increases in serum triglyceride and glucose (FIG. 2C).

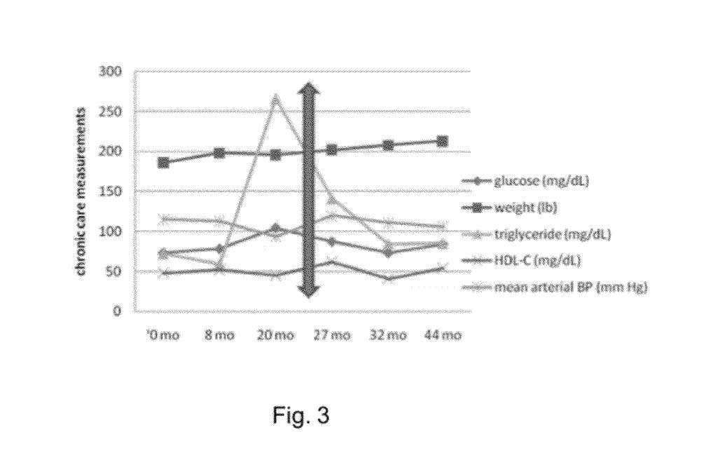

FIG. 3 shows the effects of ACE inhibitor treatment in an individual evaluated over time for glucose, body weight, triglyceride, HDL-C and mean arterial blood pressure. In the absence of an anti-lipemic agent (simvastatin, gemfibrozil, or niacin) captopril increased HDL-C, and decreased mean arterial blood pressure, serum triglyceride and glucose without causing weight loss or gain. The patient had been on atenolol (25 mg twice daily) and hydrochlorothiazide (25 mg/day), which were both discontinued at 24 months when captopril was introduced. The discontinuation of the diuretic and beta-blocker cannot fully account for the observed trends in the data. Weight increased over the 44 months of observations with BMI increasing from 26.7 kg/m2 at 0 month to 30.6 kg/m2 at 44 months.

To further evaluate the effects of ACE inhibitor, charts were screened for those not receiving lipid-lowering agents. In 81 of 105 screened charts, no known lipid-lowering agent was present. The control data (no ACE-I, no anti-lipemic agent) comprised 37 charts. The test data, i.e. with ACE-I present (but no anti-lipemic agent) comprised 44 charts. These 44 ACE-I charts included 25-300 mg/day captopril (5 charts), 10-40 mg/day lisinopril (9 charts), and 2.5-40 mg/day enalapril (30 charts).

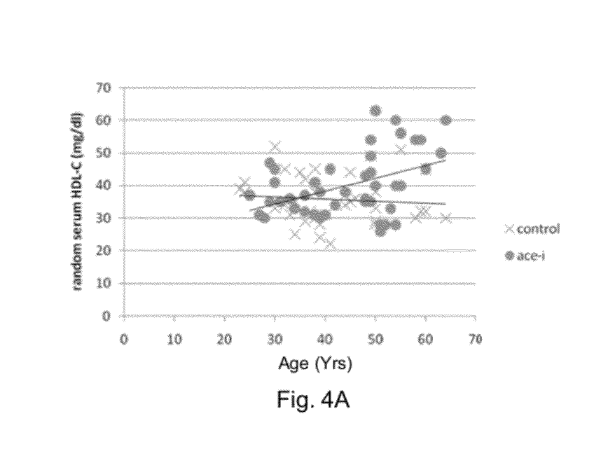

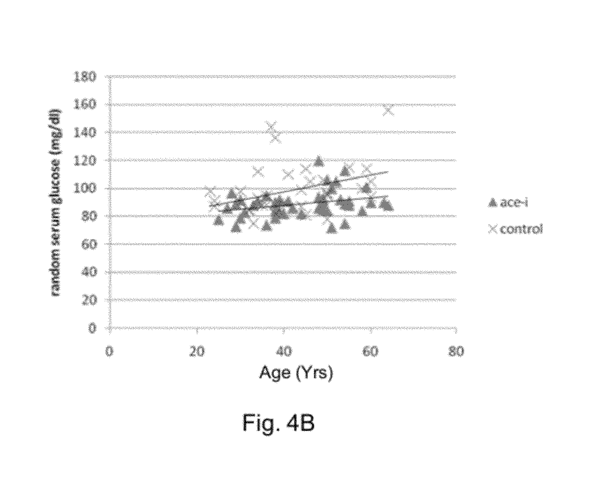

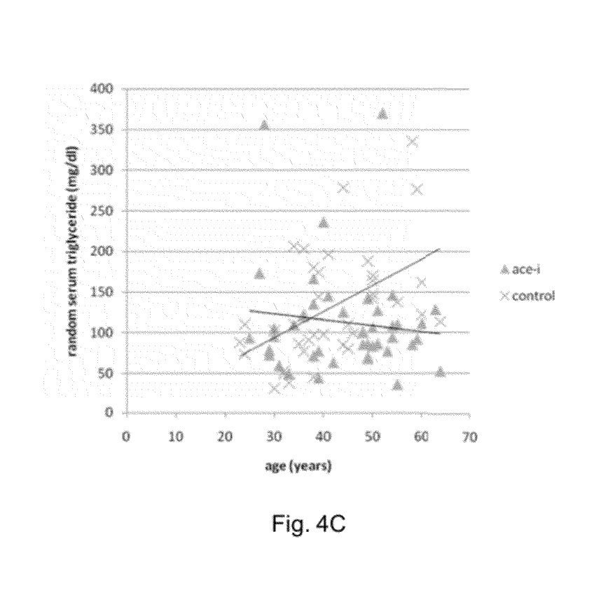

In the absence of ACE inhibitor, HDL-C did not increase with age. In fact, there was a trend for HDL-C to decrease with age. By contrast, ACE inhibitor administration significantly (P<0.02) increased HDL-C with age (FIG. 4A). Mean HDL-C was also significantly (P<0.05) higher with ACE inhibitor. When the data in FIG. 4A were averaged, means (mg/dl)+/−SEM were 35.7+/−1.2 for control, and 40.0+/−1.5 with ACE inhibitor.

Controls (no ACE inhibitor) exhibited age-related rises in random serum glucose and triglyceride (FIG. 4B, 4C). However, mean serum glucose was significantly (P<0.01) lower with ACE inhibitor. When the data in FIG. 4B were averaged, means (mg/dl)+/−SEM were 98.6+/−2.9 for control, and 89.1+/−1.5 with ACE inhibitor. ACE inhibitor significantly (P<0.01) inhibited the rise in triglyceride with age (FIG. 4C). Mean triglyceride was lower with ACE inhibitor though not significantly due to relatively high variance: means (mg/dl)+/−SEM were 131.3+/−11.4 for control and 112.6+/−10.1 with ACE inhibitor.

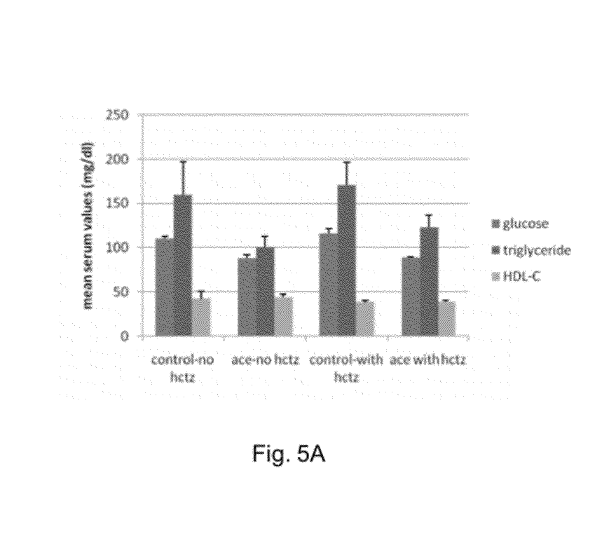

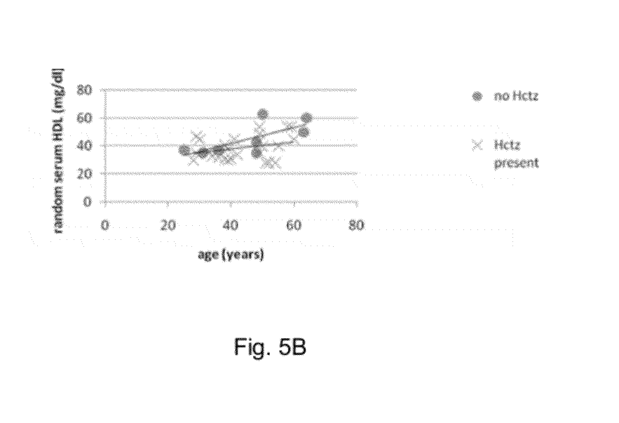

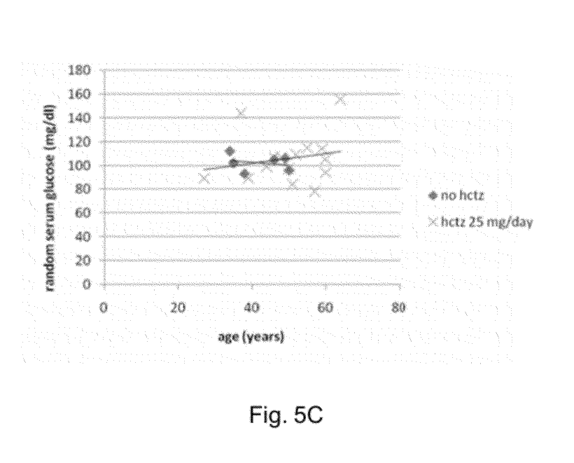

Fifty-nine (59) out of the initial 105 charts were screened for the presence Hctz. Hctz was present (25 mg/day) in 46 of 59 charts (78%), and absent in 13 (22%). Simvastatin was present in 12 of the 59 charts (20%), and gemfibrozil in 2 of 59 (3%). As shown in FIG. 5A, the insulin-resistant state was present for the 2 controls (with and without Hctz) with mean glucose >110 mg/dl, mean triglyceride >159 mg/dl, and mean HDL-C<42 mg/dl. But ACE inhibitor decreased mean glucose to 88 mg/dl and mean triglyceride to <123 mg/dl with or without Hctz. Furthermore, in the absence of Hctz, and without simvastatin being present, ACE inhibitor showed a significant age-related rise in HDL-C with a high coefficient of determination, r2=0.5 (FIG. 5B) that was much higher than that shown in FIGS. 4A (r2=0.2 with Hctz present) and 2A (r2<0.1).

In the absence of ACE inhibitor, Hctz caused an age-related increase in random serum glucose that was less apparent when Hctz was not present (FIG. 5C). However, means (mg/dl)+/−SEM for glucose, HDL-C, and triglyceride in the absence of Hctz (n=6, no ACE inhibitor, no anti-lipemic) were 102.3+/−2.8, 167.7+/−29.9, and 33.0+/−3.9, respectively. In the presence of Hctz (n=15), they were: 105.8+/−5.4, 142.9+/−18.0, and 36.1+/−2.2, respectively. There was no significant difference with or without Hctz, demonstrating that Hctz did not improve the insulin-resistant state, and may have exacerbated it.

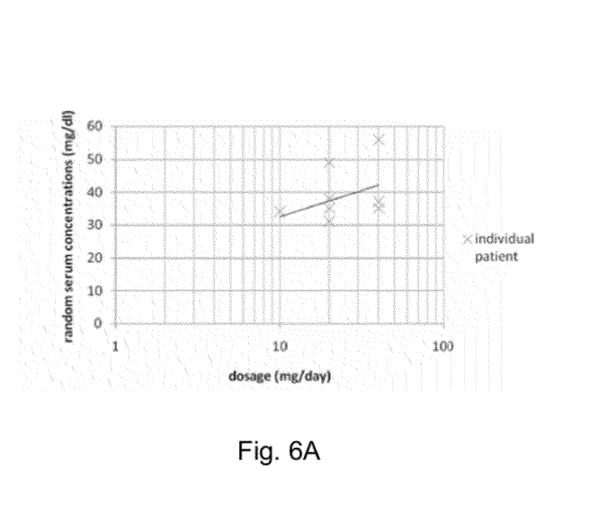

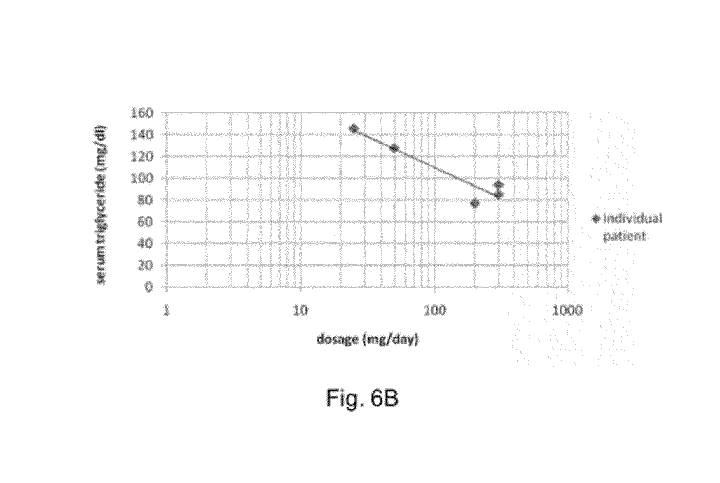

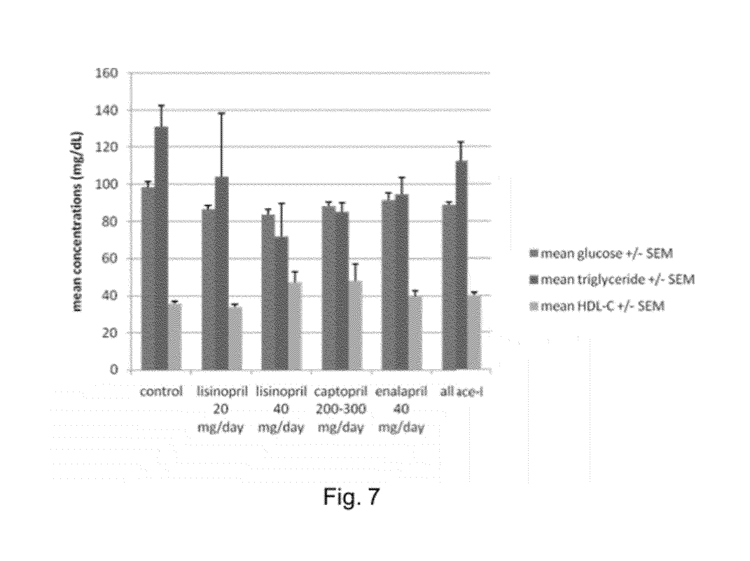

Dose-dependent effects were observed for direct-acting ACE inhibitors lisinopril and captopril. Lisinopril caused a significant (P<0.05) dose-dependent increase in HDL-C (FIG. 6A), and captopril caused a significant (P <0.02) dose-dependent decrease in triglyceride (FIG. 6B). Upper limit of normal daily dosages of lisinopril, captopril, and enalapril caused parallel decreases in mean glucose and triglyceride, and parallel increases in mean HDL-C (FIG. 7). Similar changes were observed with all ACE inhibitors as a group: 2.5-40 mg/day enalapril; 25-300 mg/day captopril; 10-40 mg/day lisinopril. Dose effects of lisinopril (20 and 40 mg/day) occurred with glucose, triglyceride and HDL-C.

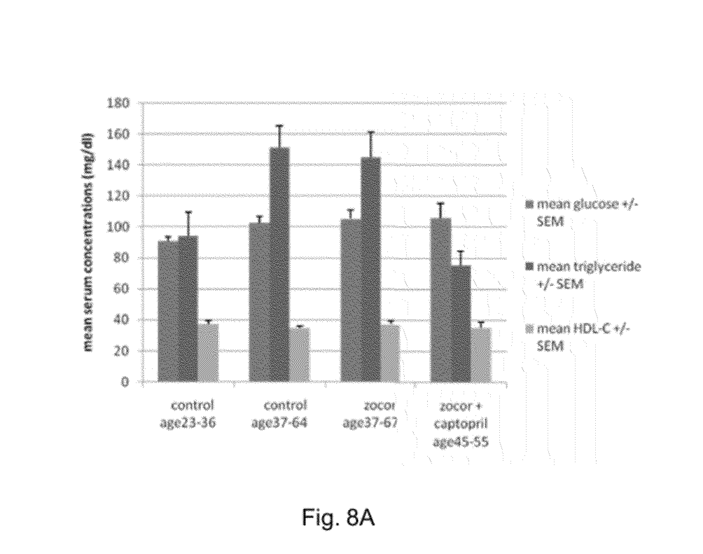

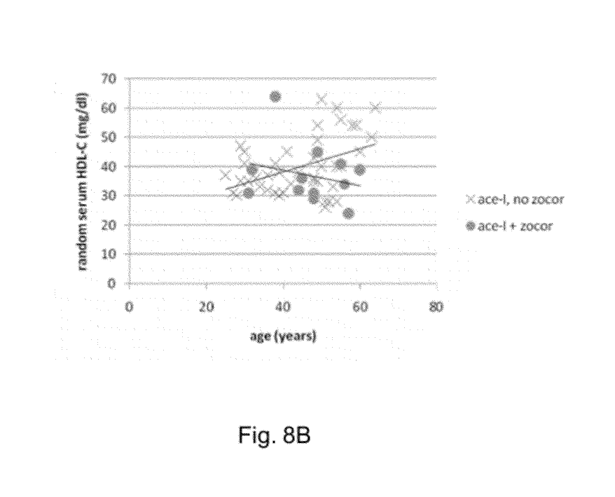

None of the original 105 charts of obese hypertensive prisoners less than 37 years old was on an anti-lipemic agent. Simvastatin was present in individuals at or beyond 37 years of age. Consequently, control data (no simvastatin, no ace-I) were analyzed with respect to 23-36 age range versus 37+ age range. As shown (FIG. 8A), the control data demonstrated an age-related insulin-resistant state at 37+ age range, in which mean glucose and triglyceride were significantly (P<0.05, P<0.01, respectively) higher at age range 37-64 compared to the younger age range 23-36. In addition, for age-matched controls (>age 37) with and without simvastatin (zocor), there was no significant difference in mean glucose, triglyceride or HDL-C.

Simvastatin was present in about 20-33% of charts. Simvastatin at 25 mg/day exacerbated the insulin-resistant state by decreasing the effect of ACE-I on glucose (FIG. 8B). Hyperglycemia persisted when ACE-I and simvastatin were present together. Simvastatin also significantly (P<0.01) decreased the effect of ACE-I on HDL-C (FIG. 8B). Hctz was not a confounding variable because it was present at similar percentages: 8 out of 13 charts (61%) for the ACE-I+ statin combination; 26 out of 44 charts (59%) for ACE-I without statin.

Simvastatin did not significantly affect random serum HDL-C with age in the patients. The regression statistics were y (HDL-C)=0.2×(years)+26.8 (n=13). 11 out of 13 charts with the combination of ACE-I+simvastatin had a mean (mg/dl)+/−SEM for total cholesterol, triglyceride, HDL-C, calculated low density lipoprotein-cholesterol (LDL-C), and glucose as follows: 152.1+/−12.2; 132.1+/−21.8; 35.1+/−1.7; 90.6+/−9.6; 98.1+/−4.0, respectively. Two (2) of the 11 had total cholesterol/triglyceride/HDL-C/LDL-C/glucose (in mg/dl) as follows: 92/126/24/43/104 and 135/170/34/67/89.

### Discussion

A unique feature of this study was that the individuals evaluated were on relatively similar diets as a result of their incarceration. Age was shown to be a factor in the onset of hyperglycemia and dyslipidemia in this study. Low HDL-C was the earliest and most sensitive indicator of the insulin-resistant state in this study, not hyperglycemia. The insulin resistant state manifested as decreased HDL-C, increased glucose and triglyceride, and was most frequent above age 37. In obese hypertensive persons with insulin resistance, the most sensitive effect of the ACE inhibitor was to increase serum HDL-C.

In this study, three different classes of ACE inhibitor drugs consistently and significantly prevented insulin resistance in obese hypertensive prisoners not on statin therapy. Consequently, the insulin-sensitizing action of ACE inhibitor is linked to inhibition of ACE.

Despite a lower in vitro dissociation constant (Ki) of enalaprilat (the active form of enalapril) when compared with captopril (Jackson, 2001), the former was less potent at preventing insulin resistance in hypertensive patients at presumptive therapeutically equivalent antihypertensive dosages. It is believed that this may be attributed to pharmacokinetic differences. Enalapril is a pro-drug requiring conversion to the active form in vivo, whereas captopril and lisinopril do not require activation to exert their pharmacodynamic effects. The demonstration of dose-dependent effects with lisinopril and captopril but not with enalapril in this study may be indicative of decreased in vivo activation of the latter in fatty livers of obese individuals in this study.

Simvastatin is a lactone pro-drug which is hydrolyzed to the active corresponding β-hydroxy acid (Mahley & Bersot, 2001). HMG-CoA reductase is the rate-limiting enzyme in cholesterol biosynthesis in the liver catalyzing the conversion of HMG-CoA to mevalonate. The hydroxy acid derivatives of statins are structural analogs of HMG-CoA, and inhibit cholesterogenesis in the liver by mimicking mevalonic acid, and competitively inhibit HMG-CoA reductase by product inhibition (Alberts et al, 1980). The resulting inhibition, through a series of additional steps, causes an increased expression of hepatic surface membrane LDL-receptors (Gaw et al, 1993). In this study, inhibition of hypertriglyceridemia by ACE inhibitor was not blocked by simvastatin, and is consistent with the known action of statins to decrease triglycerides (Ginsberg, 1998). However, for baseline triglyceride levels below 250 mg/dl, reduction of triglycerides do not exceed 25% irrespective of the dose of statin (Stein et al, 1998).

The combination of ACE inhibitor plus simvastatin yielded lower HDL-C values compared to ACE inhibitor alone. Insulin resistance is associated with low plasma HDL-C (Abate et al, 1995). It has been reported (Stone, Blum & Winslow, 1997) that high levels of HDL-C decrease the risk of coronary heart disease. HDL2-C concentrations are inversely related to the incidence of coronary atherosclerosis (Mayes & Botham, 2003). For patients with low HDL-C, it is thought that the total cholesterol/HDL ratio is predictive of coronary heart disease risk with a ratio <3.5 being favorable, and a ratio >4.5 being associated with increased risk (Castelli, 1994). The dynamic interaction between HDL-C and LDL-C with respect to coronary artery disease has yet to be elucidated. But as shown in this study, a low serum HDL-C is the earliest and most sensitive marker of the insulin-resistant state.

While certain drugs can beneficially modify levels of HDL-C and triglycerides, levels of other metabolic markers may be exacerbated. For instance, niacin increases HDL-C and decreases triglycerides but can increase serum glucose and impair control in diabetics (Garb & Grundy, 1990). Fibric acid derivatives, e.g. gemfibrozil are thought to increase HDL-C and lower triglyceride, but LDL-C rises in hypertriglyeridemic patients (Mahley & Bersot, 2001), which would include patients with insulin resistance. Metformin decreases serum triglycerides and reduces macrovascular complications of type 2 diabetes (Davis, 2006). However, metformin is not known to reduce hypertension associated with the insulin-resistant state.

The present study showed that ACE inhibitor increased HDL-C, and decreased hypertriglyceridemia and hyperglycemia in patients with demonstrated insulin resistance and metabolic syndrome. It is proposed that the principal action of the ACE inhibitor in correcting and preventing the insulin-resistant state in the patients studied is due to an ACE inhibitor-induced decrease in free fatty acid flux, resulting in a reversal of inhibitory effects of fatty acids on insulin-sensitive glucose uptake and serum HDL-C levels.

### Example 2

### Administration of ACE Inhibitor to Increase HDL-C

A non-obese 40-year old male presents for physical examination. He does not smoke, and has normal blood pressure (125/80). A family history reveals that both parents have hypertension and are type-2 diabetics. The patient's father is on anti-hypertension and anti-lipemic medications. The patient's older brother (50 years old) is obese, hypertensive, and is being treated for coronary artery disease including recent placement of a cardiac stent for primary coronary artery blockage. A lipid panel drawn on the patient reveals normal fasting glucose (100 mg/dL) and normal triglyceride (130 mg/dL) but low levels of HDL-C (30 mg/dL). Given the patients family history and low HDL-C his risk of developing one or more metabolic conditions is considered high. He is prescribed Lisinopril, 10 mg/day. After 1 year, a follow-up visit reveals that the patient's HDL-C levels have risen without concomitant weight loss or lifestyle changes.

### Example 3

### Administering ACE Inhibitor to Obese Patient

A 50-year old male presents with normal blood pressure (125/85), normal fasting glucose (105 mg/dL), and normal triglyceride (145 mg/dL). He is severely obese with a BMI of 40. A lipid panel drawn from the patient reveals low HDL-C (25 mg/dL). The patient is considered to be at significant risk of developing metabolic syndrome and/or cardiovascular disease later in life. He is advised of the risk and of the beneficial effects of losing weight. After 1 year the patient advises that despite strenuous effects to lose weight, including dieting and exercise, his bodyweight remains the same. He is prescribed Captopril, 75 mg/day. After 1 year on Captopril, the patient's HDL-C levels have risen to 30 mg/dL with his other measurements including bodyweight remaining unchanged.

1. A method for treating a patient having a metabolic condition comprising administering to said patient an effective amount of an ACE inhibitor, wherein said metabolic condition comprises at least one condition selected from the group consisting of hypertriglyceridemia, hyperglycemia, low HDL, and abdominal obesity.

2. A method as in claim 1 wherein said patient has metabolic syndrome.

3. A method as in claim 1 wherein said patient has low HDL-C.

4. A method as in claim 3 wherein said patient is obese.

5. A method as in claim 1 wherein said patient has normal blood pressure.

6. A method for treating a patient having a metabolic condition comprising administering to said patient an effective amount of an ACE inhibitor, wherein said metabolic condition comprises at least one condition selected from the group consisting of hypertriglyceridemia, hyperglycemia, low HDL, abdominal obesity, and hypertension, said method further comprising the step of measuring one or more metabolic marker prior to treatment.

7. A method of claim 6 wherein said marker is selected from glucose, triglyceride, and HDL-C.

8. A method of claim 7, further comprising the steps of measuring said one or more markers during treatment.

9. A method as in claim 8, further comprising determining an improvement in the patient's condition.

10. A method as in claim 9 wherein said determining step comprises assessing a change in the level of said marker after the onset of treatment which change trends toward normalization.

11. A method as in claim 10 wherein said change is in serum glucose.

12. A method of claim 10 wherein said change is in serum triglyceride.

13. A method of claim 10 wherein said change is in serum HDL-C.

14. A method of any one of claims 1 to 13 wherein said ACE inhibitor is selected from the group consisting of Benazepril (Lotensin®), Captopril (Capoten®), Enalapril (Vasotec®), Fosinopril (Monopril®), Lisinopril (Prinivil®, Zestril®), Moexipril (Univasc®), Perindopril (Aceon®), Quinapril (Accupril®), Ramipril (Altace®) Trandolapril (Mavik®) and Cilazapril.

15. A method of claim 14 wherein said dose is from 10 mg to 500 mg per day.

16. A method for preventing metabolic syndrome in a patient at risk thereof comprising the steps of:
a) detecting low HDL-C, and
b) administering an effective dose of an ACE inhibitor.

17. A method of claim 16 wherein said patient has normal blood pressure and further comprising the step of detecting an increase in serum HDL-C after initiation of treatment.

18. A method as in claim 6 wherein said patient has metabolic syndrome.

19. A method of claim 18 wherein said patient is obese and further comprising, after initiation of treatment, assessing i) a reduction in one or more serum markers selected from the group consisting of glucose and triglyceride and/or ii) an increase in serum HDL-C.

20. A method of claim 19 wherein said patient has normal blood pressure and the bodyweight of said patient does not change substantially during treatment.
